(19) United States
(12) Patent Application Publication
(10) Pub. No.: US 2010/0035293 A1
(43) Pub. Date: Feb. 11, 2010

(54) Pulsing of Bile Compartments in Sandwich-Cultured Hepatocytes

(76) Inventors: Kim Brouwer, Chapel Hill, NC (US); Xianbin Tian, Chapel Hill, NC (US); Peljin Zhang, Kalamazoo, MI (US); Keith Hoffmaster, Westborough, MA (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/308,138

(22) PCT Filed: Jun. 8, 2007
(86) PCT No.: PCT/US2007/013645

§ 371 Date: Oct. 9, 2009

Related U.S. Application Data
(60) Provisional application No. 60/811,816, filed on Jun. 8, 2006.

(51) Int. Cl. C12Q 1/02; C12N 5/07; C12N 5/071

(52) U.S. Cl. 435/29; 435/325; 435/354; 435/353; 435/370; 435/363; 435/350; 435/351; 435/349; 435/395; 435/401

(57) ABSTRACT

A method of pulsing cultured hepatocytes, such as sandwich-cultured hepatocytes. The method includes providing a culture of hepatocytes, the culture having at least one bile canaliculus; exposing the culture of hepatocytes to a calcium-free buffer, whereby the contents of the at least one bile canaliculus are released; and removing the calcium-free buffer Pulsing cultured hepatocytes can reduce cholestasis arid can provide an in vitro culture of hepatocytes the more closely reflects in vivo hepatocyte characteristics.

### RELATED APPLICATIONS

This application claims the benefit of U.S. Provisional Patent Application Ser. No. 60/811,816, filed Jun. 8, 2006; the disclosure of which is incorporated herein by reference in its entirety.

### TECHNICAL FIELD

The presently disclosed subject matter relates to a method of pulsing bile compartments in cultured hepatocytes, such as sandwich-cultured hepatocytes. More particularly, the presently disclosed subject matter relates to a method of pulsing a bile canaliculus or canalicular network in cultured hepatocytes.

### BACKGROUND

Bile salts are taken up into hepatocytes primarily by the sodium-dependent taurocholate co-transporter (Ntcp) and organic anion transporting polypeptides (Chandra and Brouwer, (2004) Pharm. Res. 21(5):719-735; Trauner et al., (1998) N. Engl. J. Med. 339(17):1217-1227). Organic anionic compounds including numerous drugs are taken up by sodium-independent transporters including Oatps and organic anion transporters (Oats). See, for example, Chandra and Brouwer, (2004) Pharm. Res. 21(5):719-735, and Trauner et al., (1998) N. Engl. J. Med. 339(17):1217-1227. Drugs and metabolites are cleared from hepatocytes by basolateral and canalicular efflux transport proteins, such as the multidrug resistance P-glycoproteins (Mdrs), multidrug resistance-associated proteins (Mrps) and breast cancer resistance protein (Bcrp). See, for example, Chandra and Brouwer, (2004) Pharm. Res. 21(5):719-735, and Trauner et al., (1998) N. Engl. J. Med. 339(17):1217-1227. Bile acids and bile acid metabolites are excreted into bile primarily via salt export pumps (Bsep). See, for example, Chandra and Brouwer, (2004) Pharm. Res. 21(5):719-735, and Trauner et al., (1998) N. Engl. J. Med. 339(17):1217-1227.

There is an increasing interest in in vitro cell based assays to study in vivo biological processes. Of particular interest is the development of in vitro cell based assays, or cultures, to study hepatocyte biology, such as hepatic metabolism, hepatic toxicology and hepatic biliary excretion of compounds. An in vitro hepatocyte culture that can accurately predict the in vivo metabolism, toxicity, and susceptibility to biliary excretion of new chemical entities would be of enormous value in drug discovery and development in both time and decreased use of animals.

However, in order to accurately predict an in vivo response from an in vitro system, it is desirable that the biological processes of the in vitro system are substantially reflective of in vivo biological properties. Unfortunately, current methods of culturing hepatocytes may pose problems that diminish the ability of in vitro cultures to predict in vivo responses. For example, hepatocytes cultured in a sandwich configuration form canalicular network(s) sealed by tight junctions, analogous to a closed compartment, into which bile acids and other components of bile are excreted. Due to the closed nature of the canalicular compartments, substances excreted from hepatocytes (bile and biliary constituents) accumulate in these compartments. Over multiple days in culture, this results in a cholestatic condition, wherein the bile is trapped in the bile ducts or compartments. Due to the “back-up” of the trapped bile acids and other endogenous substances the hepatocytes may attempt to compensate by up-regulation or down-regulation of various transport proteins in order to maintain homeostasis. In addition, metabolic pathways also may be affected by the degree of cholestasis, leading to an induction or inhibition of various metabolic enzymes, particularly drug metabolizing enzymes.

Cholestatic conditions in cultured hepatocytes are similar to in vivo characteristics of cholestatic related disorders and cholestatic animal models. Progressive familial intrahepatic cholestasis type 2 and 3 are caused by genetic mutations in the canalicular transport proteins BSEP (upper case denotes human) and MDR3, respectively. Dubin-Johnson syndrome is an autosomal recessive genetic disorder resulting from MRP2 mutation (Trauner et al., (1998) N. Engl. J. Med. 339(17):1217-1227). In addition to genetic defects, infection, drugs and surgery also can obstruct the excretion of bile and cause the development of cholestasis in patients. Regardless of the cause, a typical transporter expression pattern (up-regulation of Mrp3, down-regulation of Ntcp and Oatp2) is associated with currently available cholestatic animal models (Kuroda et al., (2004) J. Gastroenterol. Hepatol. 19(2):146-53; Rippin et al., (2001) Hepatology 33(4):776-782).

Existing methods have not demonstrated the ability to reduce or minimize cholestasis in cultured hepatocytes. Therefore, the ability of in vitro cultured hepatocytes to predict in vivo responses in compromised. Thus, there is a long-felt need for a method of decreasing cholestasis in cultured hepatocytes. Such a method would reduce cholestasis such that in vitro cultured hepatocytes could be used as a model to predict the in vivo metabolism of compounds of interest. Further, such a method would provide for the development of in vitro hepatocyte models to evaluate the in vivo toxicity and biliary excretion of compounds of interest (i.e., therapeutic agents).

### SUMMARY

It is an object of the presently disclosed subject matter to provide a method of pulsing cultured hepatocytes. In some embodiments, the method comprises providing a culture of hepatocytes, the culture comprising at least one bile canaliculus; exposing the culture to a calcium-free buffer, wherein the contents of the at least one bile canaliculus are released; and removing the calcium-free buffer.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE FIGURES

FIG. 1A is a light microscopy image of sandwich-cultured rat hepatocytes (SCRH) immediately following treatment with Hank's balanced salt solution (HBSS) with calcium (HBSS+Ca; left image) or calcium-free HBSS (HBSS−Ca; right image).

FIG. 1B is a light microscopy image of SCRH 12 hours following treatment with HBSS with calcium (HBSS+Ca; left image) or calcium-free HBSS (HBSS−Ca; right image).

FIG. 2A is an image of a Western blot showing the effects of pulsing SCRH once daily for 30 minutes on efflux transport protein expression. Particularly, Western blot images of efflux transport protein expression following treatment with HBSS with calcium (+) or calcium-free HBSS (−) once daily for 0, 1, 2, 4 and 6 days (−=pulsed; +=non-pulsed) are shown.

FIG. 2B is an image of a Western blot showing he effects of pulsing SCRH twice daily for 15 minutes on efflux transport protein expression. Particularly, Western blot images of efflux transport protein expression following treatment with HBSS with calcium (+) or calcium-free HBSS (−) for 0, 1, 2, 4 and 6 days (−=pulsed; +=non-pulsed) are shown.

FIG. 3A is an image of a Western blot showing the effects of pulsing SCRH once daily for 30 minutes on uptake transport protein expression. Particularly, Western blot images of uptake transport protein expression following treatment with HBSS with calcium (+) or calcium-free HBSS (−) once daily for 0, 1, 2, 4 and 6 days (−=pulsed; +=non-pulsed) are shown.

FIG. 3B is an image of a Western blot showing the effects of pulsing SCRH twice daily for 15 minutes on uptake transport protein expression. Particularly, Western blot images of uptake transport protein expression following treatment with HBSS with calcium (+) or calcium-free HBSS (−) for 0, 1, 2, 4 and 6 days (−=pulsed; +=non-pulsed) are shown.

### DETAILED DESCRIPTION

In accordance with the presently disclosed subject matter, a method is provided for the pulsing (opening and resealing the tight junctions) of a culture of hepatocytes to reduce cholestasis in the culture of hepatocytes, wherein the in vitro culture of hepatocytes more closely resembles in vivo hepatocytes compared to non-pulsed cultured hepatocytes. In some embodiments, the method comprises providing a culture of hepatocytes, the culture comprising at least one bile canaliculus; exposing the culture to a calcium-free buffer, wherein at least one bile canaliculus opens and releases the contents of at least one bile canaliculus; and removing the calcium-free buffer, wherein at least one bile canaliculus closes.

As would be appreciated by one of ordinary skill in the art, to accurately model in vivo biological processes at a desirable level, an in vitro culture of hepatocytes should be structurally and functionally similar to in vivo hepatocytes. Thus, in some embodiments of the presently disclosed hepatocyte cultures, structural and functional properties displayed by hepatocytes in vivo are established. For example, the establishment of hepatic transport systems, such as sinusoidal or canalicular transport systems, or both sinusoidal and canalicular transport systems is provided in accordance with the presently disclosed subject matter. Particularly, the establishment of at least one bile canaliculus in a hepatocyte culture is provided in accordance with the presently disclosed subject matter. A culture can comprise a plurality of bile canaliculi. The plurality of bile canaliculi can comprise a canalicular network. The establishment of at least one bile canaliculus, or canalicular network, can allow for cultured hepatocytes to excrete bile and biliary constituents into the at least one bile canaliculus, similar to biliary excretion in in vivo hepatocytes.

In addition to the canalicular transport system, establishment of specific transporters in an in vitro hepatocyte culture is provided. Exemplary transporters include, but are not limited to, Ntcp, cMoat, Oatp1, Oatp2, Mrp2, Mrp3, Pgp, Bsep and Mdr2. The expression and function of these hepatocyte transporters can be substantially similar to that seen in in vivo hepatocytes.

The establishment of normal metabolic capacity, including metabolic enzyme expression and activity, in the hepatocyte culture is also provided in accordance with the presently disclosed subject matter. Thus, the culture can comprise a metabolic capacity that is substantially reflective of in vivo hepatocyte metabolism. For example, but not limited to, the normal expression, function and activity of Phase I metabolic enzymes, such as various P450 isozymes, and Phase II metabolic enzymes, such as UDP-glucuronosyltransferases (UGT), in an in vitro hepatocyte culture are provided in the presently disclosed subject matter.

Currently, culturing hepatocytes, particularly sandwich-cultured hepatocytes, results in the accumulation of bile and biliary constituents in the biliary canaliculi between the hepatocytes. Each biliary canaliculus, which forms the biliary canaliculi or canalicular network(s), are sealed by tight junctions that form a closed compartment into which bile acids and other components of bile are excreted. In vivo, bile and biliary constituents are excreted into the canaliculi and transported to a bile duct via the canalicular network, whereby the bile and biliary constituents are removed from the hepatocytes. However, in vitro, there is no bile duct for the canaliculi or canalicular network to deposit excreted bile and biliary constituents. Therefore, this model can be described as cholestatic, since bile does not flow to the exterior, or out of, the bile canaliculi. Due to the closed nature of the canalicular compartments, bile and biliary constituents excreted from hepatocytes accumulate in these compartments. Over multiple days in culture, this results in a “back-up” of bile acids and other endogenous substances, which causes a cholestatic condition. Consequently, under a cholestatic state, the hepatocytes could attempt to compensate by up-regulating or down-regulating various transport proteins in order to maintain homeostasis of bile acids or other endogenous substances. By way of example but not limitation, hepatic transport proteins can be regulated through different mechanisms during cholestatic conditions, which can consequently result in the up-regulation of Mrp3 and down-regulation of Ntcp. In addition, metabolic pathways can also be affected by the degree of cholestasis, leading to an induction or inhibition of various metabolic enzymes.

In accordance with the presently disclosed subject matter, pulsing cultured hepatocytes is carried out to remove the bile and biliary constituents from the bile canaliculi, thereby relieving the “back-up” of bile and biliary constituents. This removal of bile and biliary constituents is analogous to the removal of bile and biliary constituents via the bile duct in vivo. Further, the application of a pulsing method of the presently disclosed subject matter can act to reduce cholestasis and maintain metabolic enzyme and transporter expression and/or function in closer approximation to in vivo levels.

In accordance with the presently disclosed subject matter, pulsing a culture of hepatocytes can be accomplished by exposing the culture of hepatocytes to a calcium-free buffer to thereby cause the release of the bile from one or more bile canaliculi. The calcium-free buffer opens the bile canaliculi by reversibly disrupting the tight junctions, thereby releasing the contents, including bile and biliary constituents, from the interior of the one or more bile canaliculi. Upon removal of the calcium-free buffer, and replacement with a buffer containing calcium, the tight junctions are reestablished and the bile canaliculi returned to their functional state. The calcium-free buffer can comprise calcium-free Hank's balanced salt solution; however, as would be appreciated by one of skill in the art upon review of the instant disclosure, any suitable calcium-free buffer, or buffer containing components designed to reduce the levels of calcium, is within the scope of the presently disclosed subject matter.

In addition to calcium-free buffers, magnesium-free buffers are also provided for pulsing a culture of hepatocytes. Also provided are buffers free of both calcium and magnesium. As would be appreciated by one of skill in the art upon review of the instant disclosure, any suitable magnesium-free or magnesium and calcium-free buffer, or buffer containing components designed to reduce the levels of magnesium and/or calcium, is within the scope of the presently disclosed subject matter.

It is further provided that the regular pulsing of the culture of hepatocytes can most effectively reduce the “back-up” of bile and biliary constituents in the bile canaliculi. Accordingly, the regular pulsing of a culture of hepatocytes can most effectively reduce cholestasis. Optionally, cultured hepatocytes are pulsed daily. However, daily pulsing of hepatocyte cultures can increase the time spent in maintaining the cells. There also is a possibility for an increased incidence of contamination since the handling of the cells is increased. Therefore, application of semi-automated systems for the maintenance of cultured cells is provided in accordance with the presently disclosed subject matter, and could reduce the costs associated with the regular pulsing of cultured hepatocytes. Further, the use of such equipment in a biological safety cabinet can lead to a decrease in the contamination.

While the following terms are believed to be well understood by one of ordinary skill in the art, the following definitions are set forth to facilitate explanation of the invention.

The term “calcium-free buffer” is meant to refer to any buffer that is substantially free of calcium. A non-limiting example of a calcium-free buffer is calcium-free Hank's balanced salt solution. As can be appreciated by one of ordinary skill in the art, any suitable buffer that is substantially free of calcium falls within the scope of the presently disclosed subject matter.

The term ‘magnesium-free buffer’ is meant to refer to any buffer that is substantially free of magnesium. As can be appreciated by one of ordinary skill in the art, any suitable buffer that is substantially free of magnesium falls within the scope of the presently disclosed subject matter.

The phrase “exposing the culture to a calcium-free buffer” is meant to refer to the contacting of the cultured hepatocytes with a substantially calcium-free buffer solution for a time sufficient to allow the opening or reversible disruption of one or more canalicular tight junctions, whereby the contents of the one or more bile canaliculi is released.

The phrases “normal metabolic function(s)”, “normal metabolic activity” and “desired metabolic characteristics” are used interchangeably herein and are meant to refer to the activity, function and/or expression of enzymes involved in metabolic pathways and metabolic reactions in a hepatocyte cell under normal basal conditions in vivo.

The phrases “desired transporter expression and function”, “normal transporter expression” and “normal transporter expression and function” are used interchangeably herein and are meant to refer to the expression and function of transporter molecules, structures and systems in a hepatocyte under normal basal conditions in vivo. Such transporters include, but are not limited to, uptake transporters Oatp1, Oatp2 and Ntcp and efflux transporters Mrp2, Mrp3, Pgp and Bsep.

The term “functional properties” includes any biological property that imparts a specified function involved in the biology of the organism, cell or biochemical reaction. In accordance with the presently disclosed subject matter, functional properties can include enzyme activity, enzyme function, enzyme expression, transporter expression and transporter function.

The term “compound”, “compound of interest” or “drug compound” are used interchangeably herein and are meant to refer to any compound wherein the characterization of the compound's metabolism, toxicity, hepatic uptake or susceptibility to biliary excretion is desirable. Exemplary compounds, compounds of interest or drug compounds include xenobiotics such as drugs and other therapeutic agents, carcinogens and environmental pollutants and endobiotics such as steroids, fatty acids and prostoglandins.

The compounds of interest that are therapeutic agents can be useful in the treatment of warm-blooded vertebrates. Therefore, the presently disclosed subject matter concerns mammals and birds.

Provided is the treatment of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, i.e., poultry, such as turkeys, chickens, ducks, geese, guinea fowl, and the like, as they are also of economical importance to humans. Thus, provided is the treatment of livestock, including, but not limited to, domesticated swine (pigs and hogs), ruminants, horses, poultry, and the like.

The phrase “evaluating a toxicological effect” is meant to refer to any suitable method of quantitatively and/or qualitatively measuring one or more toxic effects of a compound on a hepatocyte.

The term “biliary excretion” is meant to refer to a biological process wherein substances are removed from a subject's circulatory system by being taken up by hepatocytes and excreted in bile via the bile canaliculi. Uptake into the hepatocytes is mediated by transport systems endogenous to hepatocytes, including, but not limited to, Ntcp, Oatp1 and Oatp2. Excretion into the bile canaliculi is mediated by efflux transporters, including, but not limited to, Mrp2, Mrp3, Pgp and Bsep. Bile canaliculi are structures within liver tissue which receive excreted components from the hepatocytes and transport the bile to a bile duct for removal from the subject.

The presently disclosed methods of pulsing cultured hepatocytes can comprise establishing a sandwich-culture of hepatocytes wherein at least one hepatocyte layer is formed between two layers of matrix. While configuration as a sandwich-culture is the preferred configuration for the culture, any suitable configuration as would be apparent to one of ordinary skill in the art is within the scope of the presently disclosed subject matter. For example, clusters, aggregates or other associations or groupings of hepatocytes in a culture wherein at least one bile canaliculus is formed and wherein functional properties of hepatocytes are established fall within the scope of the presently disclosed subject matter. Optionally, the culture configuration facilitates the formation of a plurality of bile canaliculi reflective of in vivo hepatocytes. Also optionally, the culture configuration facilitates the formation of a canalicular network. Further, the culture configuration optionally facilitates the establishment of a culture of hepatocytes with desired metabolic characteristics substantially similar to that of in vivo hepatocytes. Likewise, desired transporter expression and function are optionally established so as to be substantially similar to that of in vivo hepatocytes.

Additionally, in a sandwich configuration, hepatocytes are cultured in monolayers between two layers of matrix or scaffolding. But, the hepatocytes can also be embedded in the matrix or can extend non-uniformly through the matrix vertically, horizontally, diagonally, or in any combination thereof, such that one-dimensional, two-dimensional and three-dimensional hepatocytes aggregates are formed. Additionally, hepatocyte cultures can be established in bioreactor systems, microenvironments or three-dimensional scaffolds, such as but not limited to, a three-dimensional flow-through system. See, for example, Griffith and Naughton, (2002) Science 295:1009-1014. Hepatocyte cultures can thus be formed by mixing hepatocyte cells with an appropriate matrix and inserting the mixture into a suitable culture container, such as a multi-well plate or culture chamber.

While collagen is a representative substrate or scaffolding for the culture of hepatocytes, any suitable substrate or scaffolding whether natural, synthetic or combinations thereof as would be apparent to one of ordinary skill in the art is within the scope of the presently disclosed subject matter. For example, other biological substrates, including but not limited to laminin and the basement membrane derived biological cell culture substrate sold under the registered trademark MATRIGEL® by Collaborative Biomedical Products, Inc. of Bedford, Mass., comprise suitable substrate or scaffolding material. Synthetic matrix materials, substrate materials or scaffolding materials, which are typically made from a variety of materials such as polymers, also fall within the scope of the presently disclosed subject matter. The variation of component materials with a particular matrix for use in culturing hepatocytes is also provided in accordance with the methods of the presently disclosed subject matter.

Any suitable source of hepatocytes as would be apparent to one of ordinary skill in the art upon review of the present disclosure is within the scope of the presently disclosed subject matter. Exemplary sources include the warm-blooded vertebrates listed above. In particular, exemplary sources include, but are not limited to, human beings, rats, mice, monkeys, apes, cats, dogs, pigs, hogs, cattle, oxen, sheep, horses, turkeys, chickens, ducks and geese.

The cultured hepatocytes can be cultured as a “long-term culture”. By “long-term culture” it is meant to refer to hepatocytes that have been cultured for at least about 12 hours. Optionally, by “long-term culture” it is meant to refer to hepatocytes that have been cultured for at least about 24 hours, for at least about 48 hours, or for at least about 72 hours. Also optionally, by “long-term culture” it is meant to refer to hepatocytes that have been cultured for at least about 96 hours. Long-term culturing facilitates the formation of bile canaliculi and the establishment of functional properties, such as metabolic pathways, within the culture.

The phrase “substantially extending the life of the culture” and/or the phrase “substantially extending the life of the hepatocytes” are used interchangeably herein and are meant to refer to extending the time the hepatocytes are cultured while maintaining functional properties substantially similar to in vivo hepatocytes. In accordance with the presently disclosed subject matter, pulsing cultured hepatocytes results in substantially reduced or minimized cholestasis, consequently reducing the confounding influences of cholestasis, such as altered transporter or metabolic function, so that the functional properties of the hepatocytes are maintained at in vivo levels for substantially longer time periods than non-pulsed cultures of hepatocytes. Alternatively, non-pulsed hepatocytes can, over the course of multiple days in culture, accumulate bile and bile constituents in the bile canaliculi, consequently resulting in compensatory changes in transporter expression and function and metabolic capacity which do not reflect the in vivo functional properties. Thus, the methods of pulsing disclosed herein serve to maintain in vivo-like functional properties in in vitro cultured hepatocytes, thereby substantially extending the life of the culture as a model of in vivo functional properties.

Following long-standing patent law convention, the terms “a” and “an” mean “one or more” when used in this application, including the claims.

### Maintenance of Metabolic Capacity Embodiment

In accordance with some embodiments of the presently disclosed subject matter, cultured hepatocytes, such as sandwich-cultured hepatocytes, are pulsed regularly, such as daily, to reduce cholestasis and the confounding influence of cholestasis, such that the metabolic capacity of the in vitro culture of hepatocytes is maintained at a level that is substantially reflective of the metabolic capacity of in vivo hepatocytes. It is provided that regularly pulsing cultured hepatocytes can reduce cholestasis such that the activity, expression and function of metabolic enzymes, such as P450 isozymes, are maintained at desired levels, wherein the desired levels are substantially similar to the metabolic enzyme activity, expression and function of in vivo hepatocytes. Further, by reducing cholestasis, the enzyme activities in the cultured hepatocytes can be maintained for extended periods of time, particularly as compared to non-pulsed hepatocytes. Of particular interest are enzymes important for the metabolism of therapeutic compounds, for example drug compounds, xenobiotics and the like.

### Maintenance of Transporter Expression and Function Embodiment

In some embodiments of the presently disclosed subject matter, the regular pulsing, such as daily, of cultured hepatocytes, such as sandwich-cultured hepatocytes, is performed to maintain hepatocyte transporter expression and function at a desired level. It is provided that a desired level of transporter expression and function is maintained at a level that is substantially similar to the expression and function of transporters of in vivo hepatocytes. By regularly pulsing the culture of hepatocytes to reduce cholestasis, transporter expression and function can be maintained for an extended period of time, particularly when compared to non-pulsed hepatocyte cultures. Exemplary transporters include, but are not limited to, Ntcp, cMOAT, OATP1, OATP2, MRP2, P-gp, BSEP and MDR2.

### Evaluation of Hepatic Toxicity Embodiment

In some embodiments of the presently disclosed subject matter, regularly pulsed, such as daily, hepatocyte cultures, such as sandwich-cultured hepatocytes, are used to evaluate the toxicological effects of a compound of interest. By regularly pulsing an in vitro culture of hepatocytes the culture will more closely mimic in vivo hepatocytes, thereby providing a superior toxicology model as compared to non-pulsed hepatocyte cultures. The application of the pulsing methods disclosed herein provide for the establishment of a non-cholestatic in vitro hepatocyte culture with a substantially extended life that closely reflects the in vivo expression and function of key transporters and metabolic enzymes. Such a hepatocyte culture can provide improved in vitro prediction of in vivo drug metabolism and toxicity, including long term toxicological effects. Further, toxicological effects of multiple compounds and/or multiple exposures can be evaluated using pulsed hepatocyte cultures. Toxicological effects can include, but are not limited to, compound-induced alterations in and/or effects on hepatocyte uptake, biliary excretion and biliary clearance.

### Evaluation of Hepatic Uptake and Excretion Embodiment

In some embodiments of the presently disclosed subject matter, regularly pulsed, such as daily, hepatocyte cultures, such as sandwich-cultured hepatocytes, can be used to evaluate the hepatic uptake and biliary excretion of compounds of interest, such as drug compounds. As disclosed in U.S. Pat. No. 6,780,580, incorporated herein by reference in its entirety, the screening of compounds of interest, e.g. therapeutic compositions, is desirable as such compounds can be taken up and excreted extensively through the biliary excretion processes whereby they have a minimal chance of imparting therapeutic effects in a subject. It is thus desirable to establish an in vitro test for a compound's susceptibility to hepatocyte uptake and biliary excretion so as to facilitate elimination of a compound with an undesirably high susceptibility from further evaluation as a therapeutic agent early in the evaluation process. Accordingly, because regularly pulsed cultures of hepatocytes maintain desired functional properties reflective of in vivo hepatocytes they provide a model for the screening of compounds of interest for susceptibility to biliary excretion.

### Reduced Cholestatic in vitro Hepatocyte Model Embodiment

In some embodiments of the presently disclosed subject matter, regularly pulsed, such as daily, hepatocyte cultures, such as sandwich-cultured hepatocytes, are used as a reduced cholestatic in vitro hepatocyte model to predict in vivo hepatic metabolism, toxicity, uptake and biliary excretion of compounds of interest, particularly drug compounds. Regular pulsing of a culture of hepatocytes can effectively reduce the “back-up” of bile and biliary constituents in the bile canaliculi to thereby reduce cholestasis, and can provide for the establishment of an in vitro culture of hepatocytes without the confounding influence of cholestasis and altered transporter or metabolic function. Accordingly, this in vitro model of in vivo hepatocyte biology can serve as a model for a wide range of hepatic research applications, as would be appreciated by one of ordinary skill in the art upon a review of the instant disclosure.

### EXAMPLES

The following examples have been included to illustrate representative modes of the invention. In light of the present disclosure, one of ordinary skill in the art will appreciate that the following examples are intended to be representative only and that numerous changes, modifications and alterations can be employed without departing from the spirit and scope of the invention.

The following examples evaluated the effect of pulsing sandwich-cultured rat hepatocytes (SCRH) with calcium-free buffer on hepatocyte morphology and cholestatic-induced modulation of transporter expression. The expression of efflux transport proteins and uptake transport proteins was evaluated. Additionally, two different pulsing treatments were assessed for their efficacy in reducing cholestasis and modulating compensatory changes in transporter expression.

### EXPERIMENTAL DESIGN FOR EXAMPLES

Freshly isolated rat hepatocytes were plated on gelled collagen coated 6-well plates at a density of 1.5 million cells/well. Cells were overlaid with a layer of gelled collagen one day after plating to form the sandwich-culture configuration. The SCRH were treated with Hank's balanced salt solution (HBSS) with calcium (HBSS+Ca) or calcium-free HBSS (HBSS−Ca) by exposing the SCRH to one of the HBSS buffers followed by removal of the buffer. Two pulsing treatments were applied: 1) incubation with HBSS−Ca for 30 minutes once per day; or 2) incubation with HBSS−Ca for 15 minutes twice per day. All treatments followed the same pre- and post- treatment procedures. The pre-treatment procedure comprised washing the SCRH with 2 ml of 37° C. HBSS+Ca or HBSS−Ca, for non-pulsed and pulsed cells, respectively. The post-treatment procedure comprised washing the SCRH with 2 ml of 37° C. HBSS+Ca and adding medium. Hepatocytes were harvested and lysed on Days 0, 1, 2, 4 and 6 using 400 ul of complete protease inhibitors in 1% SDS and 1 mM EDTA. The lysate was stored at −80° C. and later analyzed by Western blot.

### Example 1

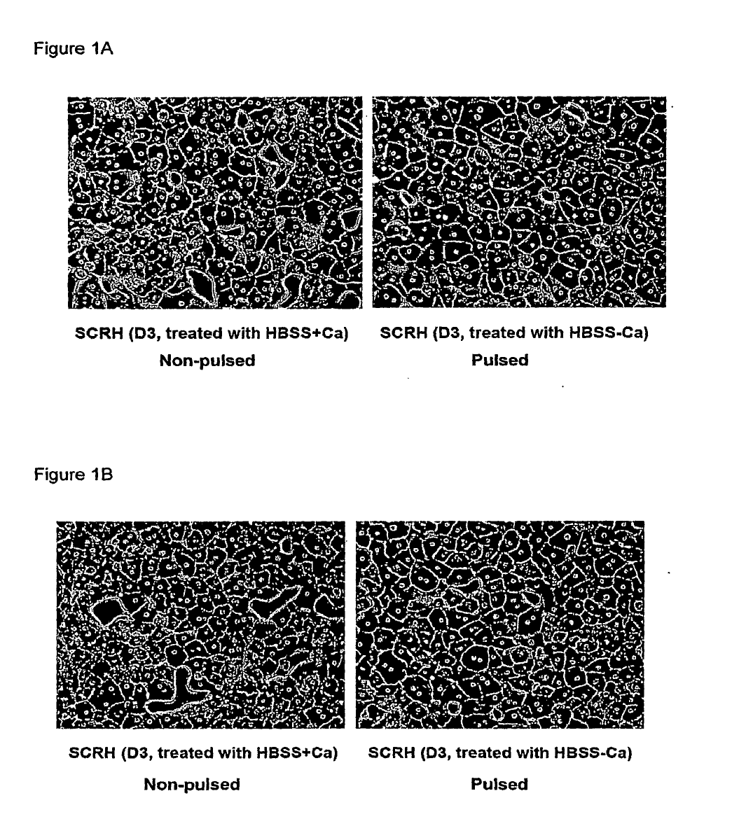

Sandwich-cultured rat hepatocytes were treated with HBSS+Ca (non-pulsed) or HBSS−Ca (pulsed). Light microscopy was used analyze the effects of pulsing on the morphology of the SCRH. Light microscopy images captured immediately after treatment show that the bile canaliculi of pulsed SCRH treated with HBSS−Ca were substantially deflated and narrowed compared to the bile canaliculi of non-pulsed SCRH treated with HBSS+Ca, indicating that the bile accumulated in the lumen of the bile canaliculi was released due to pulsing (FIG. 1A). Images captured 12 hours post-treatment illustrate that the bile canalicular network gradually resealed during the subsequent growth in culture medium, as evidenced by the substantial re-inflation of the bile canaliculi (FIG. 1B). Additionally, 12 hours post-treatment the pulsed SCRH appeared to be healthier than the non-pulsed cells (FIG. 1B), suggesting that the pulsing reduced cholestasis and the negative effects associated therewith.

### Example 2

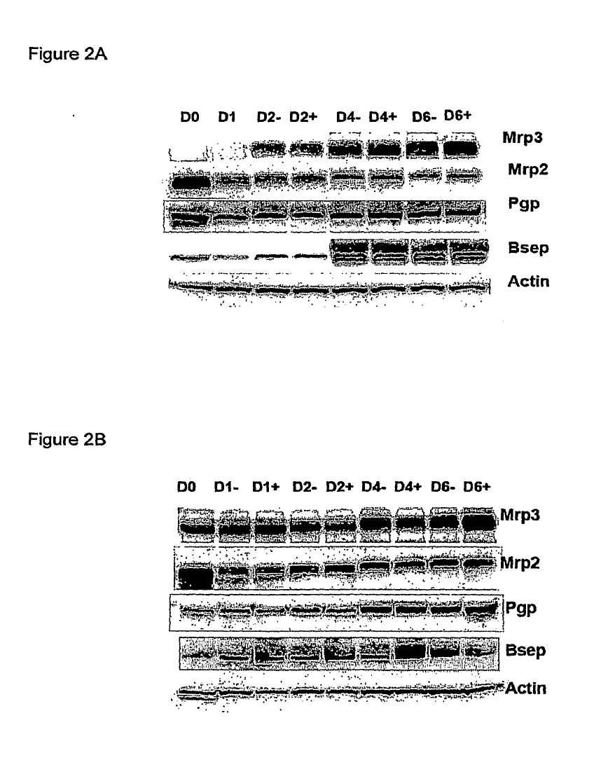

Sandwich-cultured rat hepatocytes were treated with HBSS+Ca (non-pulsed) or HBSS−Ca (pulsed) either once daily for 30 minutes (FIG. 2A) or twice daily for 15 minutes (FIG. 2B). Cells were harvested and lysed after 0, 1, 2, 4 and 6 days in culture. The expression of efflux transporters (Mrp3, Mrp2, Pgp and Bsep) was assessed by Western blot analysis. The expression of Pgp and Mrp2 appeared to be the same in non-pulsed and pulsed SCRH across all days for both pulsing treatments. The expression of Bsep appeared to be decreased on days 1, 2 and 4 in SCRH pulsed twice daily for 15 minutes (FIG. 2B), but was not affected by once daily pulsing (FIG. 2A). The expression level of the basolateral efflux transporter Mrp3 was up-regulated in non-pulsed SCRH, which is indicative of cholestasis. By day 6, this up-regulation of Mrp3 expression was attenuated by both pulsing treatments (FIGS. 2A and 2B).

This experiment demonstrates the tendency of sandwich-cultured hepatocytes to up-regulate the expression of efflux transporters, particularly Mrp3, as a compensatory mechanism against cholestasis. However, regularly pulsing the cultured hepatocytes reduced this compensatory over-expression of efflux transporters.

### Example 3

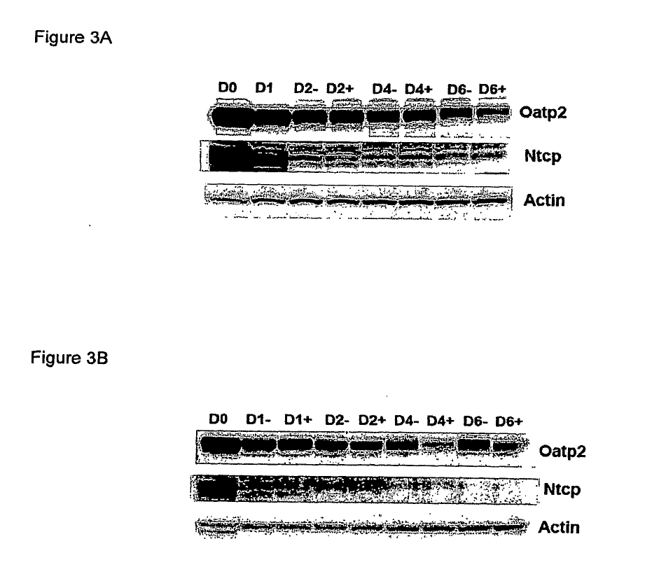

Sandwich-cultured rat hepatocytes were treated with HBSS+Ca (non-pulsed) or HBSS−Ca (pulsed) either once daily for 30 minutes (FIG. 3A) or twice daily for 15 minutes (FIG. 3B). Cells were harvested and lysed after 0, 1, 2, 4 and 6 days in culture. The expression of uptake transporters (Oatp1, Oatp2 and Ntcp) was assessed by Western blot analysis. The expression of Oatp1 and Ntcp was not affected by pulsing treatment. However, compared to non-pulsed SCRH, the expression of Oatp2 was higher on day 6 in SCRH pulsed once daily for 30 minutes (FIG. 3A) and on days 4 and 6 in SCRH pulsed twice daily for 15 minutes (FIG. 3B).

This experiment demonstrates the tendency of sandwich-cultured hepatocytes to down-regulate the expression of uptake transporters, particularly Oatp2, as a compensatory mechanism against cholestasis. However, regularly pulsing the cultured hepatocytes at least partially attenuated this compensatory down-regulation of uptake transporters.

### SUMMARY OF EXAMPLES

The periodic release of bile (pulsing) from bile canaliculi in SCRH appeared to improve the morphology of the cells. In addition, pulsing the bile canaliculi in the SCRH appeared to modulate the compensatory changes in the expression of efflux and uptake transporters due to cholestasis, thereby maintaining transporter expression at levels more representative of in vivo expression levels.

### REFERENCES

The references listed below as well as all references cited in the specification are incorporated herein by reference to the extent that they supplement, explain, provide a background for or teach methodology, techniques and/or compositions employed herein.

1. Chandra P., Brouwer K. L. (2004) Pharm. Res. 21(5):719-735.

2. Trauner M., Meier P. J., Boyer J. L. (1998) N. Engl. J. Med. 339(17):1217-1227.

3. Kuroda M., Kobayashi Y., Tanaka Y., Itani T., et al. (2004) J. Gastroenterol. Hepatol. 19(2):146-53.

4. Rippin S. J., Hagenbuch B., Meier P. J., and Stieger B. (2001) Hepatology 33(4):776-782.

5. Griffith L. G., G. Naughton. (2002) Science 295 :1009-1014.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of pulsing a culture of hepatocytes, comprising:
providing a culture of hepatocytes, the culture comprising at least one bile canaliculus;
exposing the culture to a calcium-free buffer, whereby the at least one bile canaliculus opens and releases contents of the at least one bile canaliculus; and
removing the calcium-free buffer, whereby the at least one bile canaliculus closes.

2. The method of claim 1, wherein the hepatocytes are isolated from a source selected from the group consisting of rat, mouse, human, monkey, ape, cat, dog, pig, hog, cattle, oxen, sheep, horses, turkeys, chickens, ducks and geese.

3. The method of claim 1, wherein the culture of hepatocytes further comprises a canalicular network.

4. The method of claim 1, wherein the hepatocytes are embedded in a matrix.

5. The method of claim 4, wherein the matrix is selected from the group consisting of a biological matrix medium, a synthetic matrix medium, and combinations thereof.

6. The method of claim 5, wherein the biological matrix medium is selected from the group consisting of collagens, laminins, basement membrane-derived complexes, derivatives thereof and combinations thereof.

7. The method of claim 1, wherein the culture of hepatocytes comprises a configuration selected from the group consisting of a cluster of hepatocytes, an aggregate of hepatocytes, at least one layer of hepatocytes, and combinations thereof.

8. The method of claim 1, wherein the culture of hepatocytes further comprises a sandwich-culture of hepatocytes, the sandwich-culture comprising at least one layer of hepatocytes and at least one bile canaliculus with the at least one layer of hepatocytes.

9. The method of claim 8, wherein the at least one layer of hepatocytes is sandwiched between two layers of matrix.

10. The method of claim 9, wherein the matrix is selected from the group consisting of a biological matrix medium, a synthetic matrix medium, and combinations thereof.

11. The method of claim 10, wherein the biological matrix medium is selected from the group consisting of collagens, laminins, basement membrane-derived complexes, derivatives thereof and combinations thereof.

12. The method of claim 1, wherein the method is carried out in at least one well of a multi-well plate.

13. The method of claim 1, wherein the calcium-free buffer is calcium-free Hank's balanced salt solution.

14. The method of claim 1, wherein the contents of the at least one bile canaliculus comprises bile and biliary constituents.

15. The method of claim 1, wherein pulsing the culture of hepatocytes is performed repeatedly.

16. The method of claim 1, comprising exposing the culture to a calcium-free buffer at least once per day.

17. The method of claim 1, comprising exposing the culture to a calcium-free buffer at least twice per day.

18. The method of claim 1, wherein pulsing the culture of hepatocytes substantially improves the ability of the cultured hepatocytes to maintain normal transporter expression and metabolic functions that more closely reflect the transporter expression and metabolic functions of hepatocytes in vivo compared to non-pulsed cultured hepatocytes.

19. The method of claim 1, wherein pulsing the culture of hepatocytes substantially extends the life of the hepatocyte culture compared to non-pulsed cultured hepatocytes.

20. The method of claim 1, wherein the opening of the at least one bile canaliculus comprises disrupting tight junctions of the at least one bile canaliculus.

21. The method of claim 1, wherein the culture of hepatocytes is exposed to a magnesium-free buffer.

22. A culture of hepatocytes made by the method of claim 1.

23. A culture of hepatocytes made by the method of claim 8.

24. A method of maintaining desired hepatic metabolic characteristics in an in vitro culture of hepatocytes, the method comprising:
providing a culture of hepatocytes, the culture comprising at least one bile canaliculus and having substantially normal metabolic activity; and
pulsing the culture of hepatocytes, the pulsing comprising:
exposing the culture to a calcium-free buffer, whereby the at least one bile canaliculus opens and releases the contents of the at least one bile canaliculus; and
removing the calcium-free buffer, whereby the at least one bile canaliculus closes,
whereby the desired metabolic characteristics of the in vitro cultured hepatocytes are maintained.

25. The method of claim 24, wherein the culture of hepatocytes further comprises a sandwich-culture of hepatocytes, the sandwich-culture comprising at least one layer of hepatocytes sandwiched between two layers of matrix and at least one bile canaliculus with the at least one layer of hepatocytes.

26. The method of claim 24, wherein pulsing the culture of hepatocytes is performed repeatedly.

27. The method of claim 24, comprising exposing the culture to a calcium-free buffer at least once per day.

28. The method of claim 24, comprising exposing the culture to a calcium-free buffer at least twice per day.

29. The method of claim 24, wherein the metabolic characteristics include Phase I metabolic activities.

30. The method of claim 29, wherein the Phase I metabolic activities include P450 isozyme activity.

31. The method of claim 24, wherein the metabolic characteristics include Phase II metabolic activities.

32. The method of claim 31, wherein Phase II metabolic activities include UDP-glucuronosyltransferase activity.

33. The method of claim 24, comprising screening compounds for susceptibility to biliary excretion using the culture of hepatocytes.

34. A method of maintaining desired hepatic transporter expression and function in an in vitro culture of hepatocytes, the method comprising:
providing a culture of hepatocytes, the culture comprising at least one bile canaliculus and having substantially normal transporter expression and function; and
pulsing the culture of hepatocytes, the pulsing comprising:
exposing the culture to a calcium-free buffer, whereby the at least one bile canaliculus opens and releases contents of the at least one bile canaliculus; and
removing the calcium-free buffer, whereby the at least one bile canaliculus closes,
whereby the expression and function of transporters in the in vitro cultured hepatocytes are maintained at a desired level.

35. The method of claim 34, wherein the culture of hepatocytes further comprises a sandwich-culture of hepatocytes, the sandwich-culture comprising at least one layer of hepatocytes sandwiched between two layers of matrix and at least one bile canaliculus with the at least one layer of hepatocytes.

36. The method of claim 34, wherein pulsing the culture of hepatocytes is performed repeatedly.

37. The method of claim 34, comprising exposing the culture to a calcium-free buffer at least once per day.

38. The method of claim 34, comprising exposing the culture to a calcium-free buffer at least twice per day.

39. The method of claim 34, comprising screening compounds for susceptibility to biliary excretion using the culture of hepatocytes.

40. The method of claim 34, wherein the transporters are efflux transporters selected from the group consisting of Mrp2, Mrp3, Pgp and Bsep.

41. The method of claim 34, wherein the transporters are uptake transporters selected from the group consisting of Oatp1, Oatp2 and Ntcp.

42. A method of evaluating hepatic drug toxicity in an in vitro culture of hepatocytes, the method comprising:
providing a culture of hepatocytes, the culture comprising at least one bile canaliculus;
pulsing the culture of hepatocytes, the pulsing comprising:
exposing the culture to a calcium-free buffer, whereby the at least one bile canaliculus opens and releases contents of the at least one bile canaliculus; and
removing the calcium-free buffer, whereby the at least one bile canaliculus closes,
exposing the culture of hepatocytes to at least one drug compound at least once; and
evaluating a toxicological effect of the exposure of the at least one drug compound on the culture of hepatocytes,
wherein the toxicological effect of the exposure to the at least one drug compound on the pulsed in vitro culture of hepatocytes more closely predicts the toxicological effects of in vivo hepatocytes compared to non-pulsed in vitro cultured hepatocytes.

43. The method of claim 42, wherein the culture of hepatocytes further comprises a sandwich-culture of hepatocytes, the sandwich-culture comprising at least one layer of hepatocytes sandwiched between two layers of matrix and at least one bile canaliculus with the at least one layer of hepatocytes.

44. The method of claim 42, wherein pulsing the culture of hepatocytes is performed repeatedly.

45. The method of claim 42, comprising exposing the culture to a calcium-free buffer at least once per day.

46. The method of claim 42, comprising exposing the culture to a calcium-free buffer at least twice per day.

47. The method of claim 42, wherein the culture of hepatocytes is exposed to a plurality of drug compounds.

48. The method of claim 42, wherein the culture of hepatocytes is repeatedly exposed to one or more drug compounds.

49. The method of claim 42, wherein the pulsing substantially extends the life of the culture of hepatocytes as compared to non-pulsed cultured hepatocytes, such that long-term toxicological effects can be evaluated.

50. A reduced cholestatic in vitro hepatocyte model for modeling in vivo hepatocyte metabolism and drug toxicity, comprising:
a culture of hepatocytes having at least one bile canaliculus, wherein the culture of hepatocytes is regularly pulsed to reduce cholestasis of the culture of hepatocytes, the pulsing of the culture of hepatocytes comprising exposing the hepatocytes to a calcium-free buffer whereby contents of the at least one bile canaliculus are released.

51. The hepatocyte model of claim 50, wherein the hepatocytes are isolated from a source selected from the group consisting of rat, mouse, human, monkey, ape, cat, dog, pig, hog, cattle, oxen, sheep, horses, turkeys, chickens, ducks and geese.

52. The hepatocyte model of claim 50, wherein the culture of hepatocytes further comprises a canalicular network.

53. The hepatocyte model of claim 50, wherein the hepatocytes are embedded in a matrix.

54. The hepatocyte model of claim 51, wherein the matrix is selected from the group consisting of a biological matrix medium, a synthetic matrix medium, and combinations thereof.

55. The hepatocyte model of claim 54, wherein the biological matrix medium is selected from the group consisting of collagens, laminins, basement membrane-derived complexes, derivatives thereof and combinations thereof.

56. The hepatocyte model of claim 50, wherein the culture of hepatocytes comprises a configuration selected from the group consisting of a cluster of hepatocytes, an aggregate of hepatocytes, at least one layer of hepatocytes, and combinations thereof.

57. The hepatocyte model of claim 50, wherein the culture of hepatocytes further comprises a sandwich-culture of hepatocytes, the sandwich-culture comprising at least one layer of hepatocytes and at least one bile canaliculus with the at least one layer of hepatocytes.

58. The hepatocyte model of claim 57, wherein the at least one layer of hepatocytes is sandwiched between two layers of matrix.

59. The hepatocyte model of claim 58, wherein the matrix is selected from the group consisting of a biological matrix medium, a synthetic matrix medium, and combinations thereof.

60. The hepatocyte model of claim 59, wherein the biological matrix medium is selected from the group consisting of collagens, laminins, basement membrane-derived complexes, derivatives thereof and combinations thereof.

61. The hepatocyte model of claim 50, wherein the hepatocytes are cultured in at least one well of a multi-well plate.

62. The hepatocyte model of claim 50, wherein the calcium-free buffer is calcium-free Hank's balanced salt solution.

63. The hepatocyte model of claim 50, wherein the contents of the at least one bile canaliculus comprises bile and biliary constituents.

64. The hepatocyte model of claim 50, wherein pulsing the culture of hepatocytes is performed repeatedly.

65. The hepatocyte model of claim 50, comprising exposing the culture to a calcium-free buffer at least once per day.

66. The hepatocyte model of claim 50, comprising exposing the culture to a calcium-free buffer at least twice per day.

67. The hepatocyte model of claim 50, wherein the model can be used to screen compounds for susceptibility to biliary excretion.

68. A reduced cholestatic hepatocyte model kit, comprising:
isolated hepatic cells;
growth media; and
a calcium-free buffer.

69. The kit of claim 68, wherein the hepatocytes are isolated from a source selected from the group consisting of rat, mouse, human, monkey, ape, cat, dog, pig, hog, cattle, oxen, sheep, horses, turkeys, chickens, ducks and geese.

70. The kit of claim 68, wherein the calcium-free buffer is calcium-free Hank's balanced salt solution.

71. The kit of claim 68, further comprising instructions for culturing and pulsing the hepatocytes.
